(19) United States
(12) Patent Application Publication
(10) Pub. No.: US 2017/0112418 A1
(43) Pub. Date: Apr. 27, 2017

(54) MOTION CAPTURE AND ANALYSIS SYSTEM FOR ASSESSING MAMMALIAN KINETICS

(71) Applicant: KINETISENSE INC., Medicine Hat (CA)

(72) Inventors: Ryan Comeau, Medicine Hat (CA); Evangelos Pterneas, Medicine Hat (CA); David Schnare, Medicine Hat (CA)

(73) Assignee: KINETISENSE INC., Medicine Hat, AB (CA)

(21) Appl. No.: 15/128,048

(22) PCT Filed: Mar. 23, 2015
(86) PCT No.: PCT/CA2015/050220

§ 371 Date: Sep. 21, 2016

Related U.S. Application Data
(60) Provisional application No. 61/969,016, filed on Mar. 21, 2014.

(51) Int. Cl. A61B 5/11; A61B 5/0205; A61B 3/11; A61B 5/0496; A61B 5/02; A61B 5/0402; A61B 5/0476; A61B 5/0488; A61B 5/00; A61B 3/113

(52) U.S. Cl. CPC A61B 5/1128; A61B 5/0077; A61B 5/0075; A61B 5/1124; A61B 5/486; A61B 5/749; A61B 5/4836; A61B 5/7282; A61B 5/0205; A61B 3/112; A61B 3/113; A61B 5/02028; A61B 5/4519; A61B 5/4542; A61B 5/0402; A61B 5/0476; A61B 5/0488; A61B 5/0496; A61B 5/1102; A61B 5/0002; A61B 5/021

(57) ABSTRACT

Computer-implemented methods and systems for con-currently capturing video imagery with a 3D infrared camera, an audio commentary, and at least one set of physiological data during a mammalian subject's performance of one or more selected musculoskeletal movements for use in assessments of their range of motions.

### TECHNICAL FIELD

The present disclosure pertains to motion analysis, and more particularly to computer-implemented methods for range-of-motion data capture and analysis for assessment of mammalian kinetics.

### BACKGROUND

Use of video recordings for analyses of movements of human subjects has been gaining popularity for a variety of applications including, for example, providing immediate feedback to subjects during their performance of exercises as teaching aids for the training and execution of proper ranges of motions of their appendages and body motions. Other applications include use for monitoring and recording physical therapy programs and charting changes in patients' ranges in motion over treatment time courses. Other applications use for diagnosing neurological related effects on balance and gait and subsequent responses to therapeutic treatments. Other applications include use for training subjects in suitable range of motions to avoid motion-related workplace injuries.

However, there are many shortcomings associated with the video recording and assessment methods currently available. For example, many such methods require subjects to wear one or more markers to enable video capture of selected appendages during the performance of range of motion movements. It is difficult to ensure that the markers are placed in the precisely the same location during subsequent discrete video recording events, and therefore, comparisons of multiple records of the subject's ranges of motion movements have inherent large variability. Another problem is that it has been shown that markers placed into contact with and or adhered to a subject's body may cause alterations in muscle activity signals thereby resulting in inaccurate data pertaining to the subject's ranges of motion movements.

Another problem is that a subject's physiological data and their verbal feedback are not recorded during their performance of the range of motions movements, limiting a clinician's assessments to observable changes in ranges of motions in combination with their consideration of the subject's general comments regarding their comfort levels in performing the movements.

### SUMMARY

The exemplary embodiments of the present disclosure generally relate to computer-implemented methods and systems for capturing, analyzing, and reporting assessments of a mammalian subject's range of motions of selected musculoskeletal movements.

The computer-implemented methods generally comprise the steps of:
1. arranging a 3D infrared video camera to capture a view of a mammalian subject's selected appendage, alternatively a view of a selected joint, alternatively a view of a selected body portion, alternatively a view of the entire body;
2. providing instructions to the subject to: (i) perform a specific range of motion movement with the selected appendage, the alternatively joint, alternatively the body portion, alternatively the entire body, and (ii) provide a verbal commentary during their performance of the selected range of motion movement;
3. recording with a 3D infrared camera the subject's performance of the selected range of motion movement while concurrently recording the subject's verbal commentary during their performance of the movement;
4. concurrently recording selected physiological data exemplified by blood pressure, heart rate, ECG and/or BCG data, breathing rates, blood oxygen levels, blood lactic acid levels, and the like,
5. storing the visual, audio, and physiological data recordings electronically as a set of discrete range of motion event data records;
6. performing a kinesiology assessment of the subject's set of discrete range of motion event data records, producing a report detailing and summarizing the range of motion movement characteristics, and assigning an unique identification code for the set of data records collected, the assessment of the data records, and the report produced for the discrete range of motion event data records and assessment;
7. repeating steps 3 and 4 at selected time periods to generate a plurality of sets of discrete range of motion event data records; and
8. comparing the assessments from the subject's plurality of sets of discrete range of motion event data records to produce one or more progress reports.

According to one aspect, the computer-implemented methods disclosed herein are applied for the purposes of diagnosis of pain or alternatively, for determination of changes in mobility limits and/or flexibility over selected periods of time.

According to another aspect, the computer-implemented methods disclosed herein are applied for the purposes of assessments of changes in a subject's selected range of motion movements during rehabilitation of injured joints and/or other musculoskeletal body parts, alternatively atrophied joints and/or body parts.

According to another aspect, the computer-implemented methods disclosed herein are applied for the purposes of monitoring changes in a subject's selected range of motion movements during physical training for improving the subject's performance in a selected sport or alternatively, in the execution of a physical task.

The exemplary systems for use with the exemplary computer-implemented methods disclosed herein generally comprise at least one infrared 3D video camera, a microphone, a software program for recording, assessing, reporting, and storing imagery captured by a 3D infrared video camera with concurrently captured audio records from the subject. It is optional for the systems to additionally comprise two or more infrared video camera for concurrent capture of a subject's selected range of motion movements from three dimensions, e.g., from their side profile, from their front or their back, and from overhead (i.e., in the X, Y, and Z axes). The exemplary systems may additionally comprise instruments for concurrently capturing various physiological indices such as blood pressure, heart rate, ECG data, BCG data, breathing rates, blood oxygen levels, blood lactic acid levels, and the like.

Data processing systems for implementing the methods, and computer program products comprising tangible computer readable media embodying instructions for implementing the methods, are provided.

### DESCRIPTION OF THE DRAWINGS

These and other features will become more apparent from the following description in which reference is made to the appended drawings wherein:

FIG. 1 is a flow chart showing a first exemplary computer-implemented method for assessing a subject's temporomandibular jaw range of motion movements;

FIG. 2 is an exemplary sample panel that is completed by a dental services practitioner for defining a set of data records collected during the subject's performance of a range of motion movements;

FIG. 3 is an exemplary sample panel that is completed by a dental services practitioner during their assessment of the set of data records collected during the subject's performance of a range of motion movements;

FIG. 4 is a flowchart showing a second exemplary computer-implemented method for post-injury assessment of a subject's range of motion movements of a selected body portion;

FIG. 5 is an exemplary sample panel that is completed by a musculoskeletal therapist practitioner for defining a set of data records collected during the subject's performance of a range of motion movements pertaining to a post-injury trauma;

FIG. 6 is an exemplary panel that is completed by a musculoskeletal therapist practitioner during their assessment of the set of data records collected during the subject's post-injury performance of a range of motion movements;

FIG. 7 is an exemplary sample panel that is complete by a musculoskeletal therapist practitioner during their assessment of a plurality of sets of data records collected during the subject's post-injury performance of a range of motion movements;

FIG. 8 is a flowchart showing a third exemplary computer-implemented method for assessment of a subject's range of motion movements relating to their posture;

FIG. 9 is an exemplary sample panel that is completed by a musculoskeletal therapist practitioner for defining a set of data records collected during the subject's performance of a range of motion movements pertaining to their posture;

FIG. 10 is an exemplary sample panel that is completed by a dental services practitioner during their assessment of the set of data records collected during the subject's performance of a range of motion movements pertaining to their posture; and

FIG. 11 is a flowchart showing a fourth exemplary computer-implemented method for assessment of a subject's range of motion movements pertaining to sports training.

### DETAILED DESCRIPTION

The exemplary embodiments of the present disclosure generally relate to computer-implemented methods and systems for recording, assessing, reporting, and storing: (i) imagery of a mammalian subject's range of motion movements captured by a 3D infrared video camera, with (ii) concurrently captured audio records of the subject's verbal comments during performance of the range of motion movements with concurrently detected, and (iii) physiological data collected while the mammalian subject is performing the selected range of motion movements.

The exemplary systems disclosed herein generally comprise at least one three-dimensional (3D) infrared video camera for recording a subject's range of motion movements, a microphone for recording the subject's vocal commentary during their performance of the range of motion movements, one or more instruments for detecting and collecting selected physiological data generated by the subject while they are performing the range of motion movements, and a microprocessor in communication with a computer-implemented software program for receiving, processing, correlating, reporting, and storing data from each of the 3D infrared camera, microphone, physiological data collection instruments.

A particularly suitable 3D infrared camera for use in the exemplary systems disclosed herein is MICROSOFT®'s KINECT 3S infrared camera from MicroSoft (MICROSOFT and KINECT are registered trademarks of the MicroSoft Corp., Redmond, Wash., USA). Other suitable 3D infrared cameras are exemplified by INTEL®'s REALSENSE® 3D camera (INTEL and INTEL REALSENSE are registered trademarks of the Intel Corp., Santa Clara, Calif., USA) and PANASONIC's LUMIX® 3D stereo camera (PANASONIC and LUMIX are registered trademarks of Panasonic Corp., Secaucus, N.J., USA).

Suitable instruments for detecting and capturing physiological data are exemplified by heart rate monitors; blood pressure monitors; VO2 monitors comprising an oxygen analyzer, a carbon dioxide analyzer, and a ventilometer (also commonly referred to as a respirometer) or alternatively a pnuemotachometer; pulse oximeters for measuring oxygen saturation in the vascular system; lactic acid monitors; galvanic skin response (GSR) monitors;

electrocardiograph (ECG) monitors and ballistocardiograph (BCG) monitors for monitoring cardiac activity; electroencephalograph (EEG) monitors for monitoring brain activity, electromyography (EMG) modules for monitoring muscular activity and or electrical activity in selected muscles; electrooculograph modules for monitoring eye movements; bite force meters, and the like.

Suitable microprocessors are exemplified by laptop computers, desktop computers, tablets, and mainframe computers.

The computer-implemented methods of the present disclosure generally comprise modules for recording a set of imagery data, audio records, and selected physiological data for a selected range of motion movements; for processing the imagery data, audio records, and physiological data; for correlating the data; for assessing the correlated data; for summarizing and reporting the data, and for storing the data. The computer-implemented methods of the present disclosure additionally comprise modules for comparing, correlating, and assessing a plurality of sets of imagery data, audio records, and selected physiological data separately collected during two or more spaced-apart events for recording the selected range of motion movements; and for summarizing, reporting, and storing the correlations and assessments.

The computer-implemented methods and related systems disclosed herein enable health services practitioners such as those exemplified by chiropractors, kinesiologists, orthopaedic specialists, orthotists, prosthetists, physiotherapists, massage therapists, dentists, and the like, to integrate multiple types of data into their assessment of a subject's range of motion movements during a testing event and optionally, over a series of spaced-apart testing events. The computer-implemented methods and related systems disclosed herein are particularly suitable for assessments of a dysfunctional component of the subject's musculoskeletal system, development of a diagnosis of the dysfunction, development of a treatment plan for providing a therapy for the dysfunction, and for monitoring the subject's progress over time in response to the therapeutic treatment plan.

The computer-implemented methods and related systems disclosed herein are particularly suitable for assessment of a dysfunction or alternatively an injury or alternatively an atrophy or alternatively a normal pain-free range of motion in one or more of a subject's ankle joints, knee joints, hip joints, wrist joints, elbow joints, shoulder joints, lower back, upper back, neck, jaw joints, and the like. The present computer-implemented methods and related systems are also suitable for assessment of a subject's muscles and muscle groups such as those exemplified by lower leg muscles, upper leg muscles including hamstrings and glutes, finger muscles, hand muscles, wrist muscles, lower arm muscles, upper arm muscles, shoulder muscles, lower back muscles, upper back muscles, neck muscles, jaw muscles, and the like. The present computer-implemented methods and related systems are also suitable for assessment of a subject's repetitive work-related or sport-related movements for the purposes of providing injury avoidance training or alternatively performance improvement training or alternatively posture improvement training.

The computer-implemented methods of the present disclosure generally comprise the steps of:
1. arranging a 3D infrared video camera to capture a view of a mammalian subject's selected appendage, alternatively a view of a selected joint, alternatively a view of a selected body portion, alternatively a view of the entire body;
2. providing instructions to the subject to: (i) perform a specific range of motion movement with the selected appendage, the alternatively joint, alternatively the body portion, alternatively the entire body, and (ii) provide a verbal commentary during their performance of the selected range of motion movement;
3. recording with a 3D infrared camera the subject's performance of the selected range of motion movement while concurrently recording the subject's verbal commentary during their performance of the movement. For example, the subject's verbal commentary may include a statement at the exact time a discomfort or pain is first experienced and/or a rating of the discomfort or pain on a scale of 1 wherein 1 represents detection of the discomfort or pain and wherein 10 represents excruciating pain;
4. concurrently recording selected physiological data exemplified by blood pressure, heart rate, ECG and/or BCG data, breathing rates, blood oxygen levels, blood lactic acid levels, and the like,
5. storing the visual, audio, and physiological data recordings electronically as a set of discrete range of motion event data records;
6. performing a kinesiology assessment of the subject's set of discrete range of motion event data records, producing a report detailing and summarizing the range of motion movement characteristics, and assigning an unique identification code for the set of data records collected, the assessment of the data records, and the report produced for the discrete range of motion event data records and assessment;
7. repeating steps 3 and 4 at selected time periods to generate a plurality of sets of discrete range of motion event data records; and
8. comparing the assessments from the subject's plurality of sets of discrete range of motion event data records to produce one or more progress reports.

The computer-implemented methods disclosed herein enable the concurrent recording of imagery data of a subject's selected range of motion movements by one or more 3D infrared cameras with audio recording of commentaries by the subject during their performance of the selected range of motion movements along with at least one recording of physiological data generated by the subject's body during their performance of the selected range of motion movements. It is well-known that the thresholds for detection of discomfort and pain vary considerably among individuals, and while it is useful for a subject to verbally indicate when discomfort and pain are experienced, it is preferable from a practitioner's perspective to also include physiological data collected from the subject for assessment with the imagery and audio records collected during the subject's performance of the selected range of motion movements. Accordingly, the computer-implemented methods disclosed herein include collection of at least one type of physiological data concurrent with the recording of imagery data and audio data during a range of motion testing event. The scope of the present disclosure encompasses the collection of two or more types of physiological data concurrent with the recording of imagery data and audio data during a range of motion testing event. The type(s) of physiological data recorded may be selected for its (their) suitability to provide information to a health services practitioner that directly relates to the subject's physiological responses during their performance of the selected range of motion movements.

Suitable physiological data that may be concurrently recorded with imagery data and audio data during a range of motion testing event are exemplified by muscular activity, particularly relating to weight shifting and/or muscular force shifting during motion, heart rates, blood pressure, oxygen saturation in the vascular system, carbon dioxide levels in the vascular system, lactic acid levels in the vascular system, respiration rates, skin galvanic responses, cardiac activity, eye movements, eye dilation, bite force, among others.

The computer-implemented methods disclosed herein enable a practitioner to precisely position a subject prior to commencing a second range of motion recording event in reference to the subject's starting position during the first recording event for their selected range of motion movements, using visual cues from the imagery data collected during the first recording event. Changes in the subject's range of motion movements during the second recording event and subsequent recording events, can be compared and assessed in reference to the imagery data, audio data, and physiological data recorded during the first recording event for the selected range of motion movements.

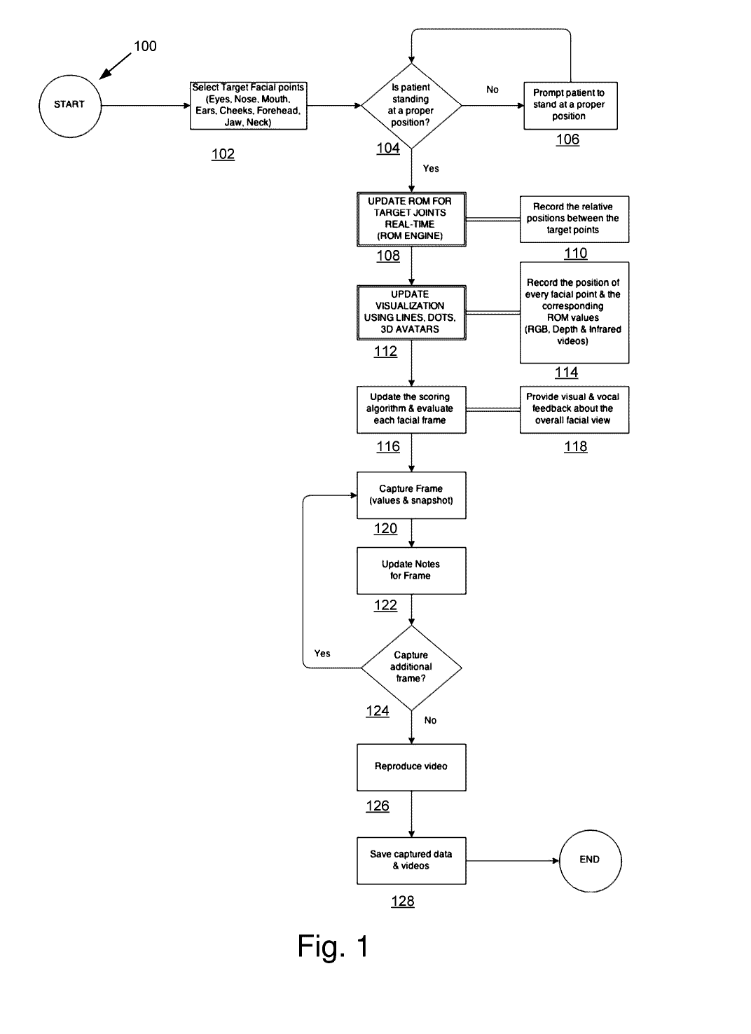

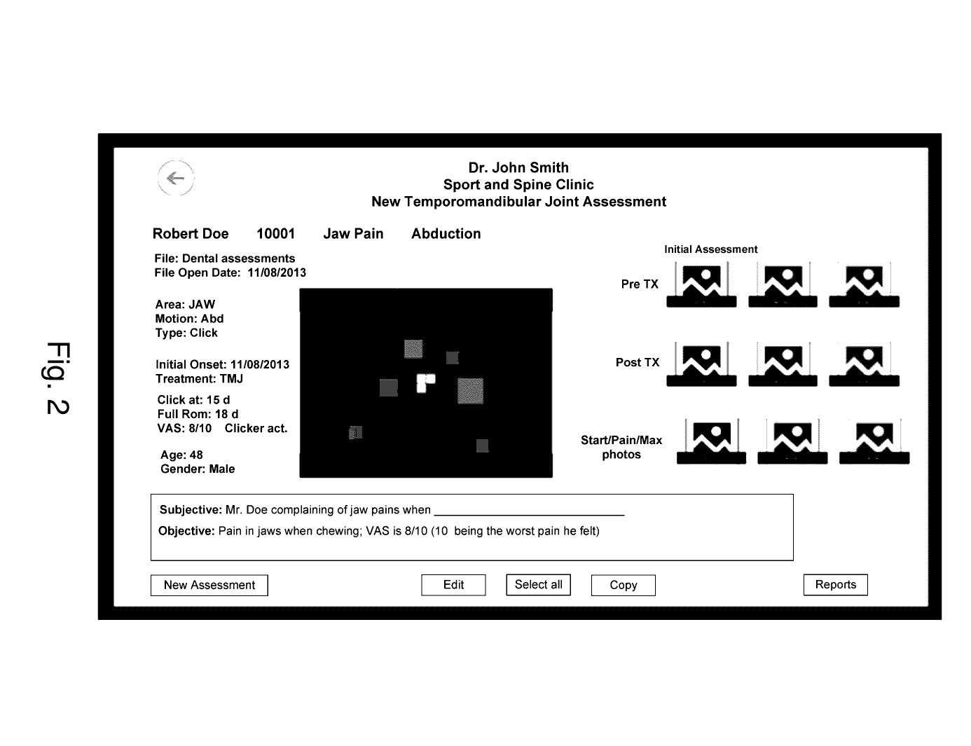

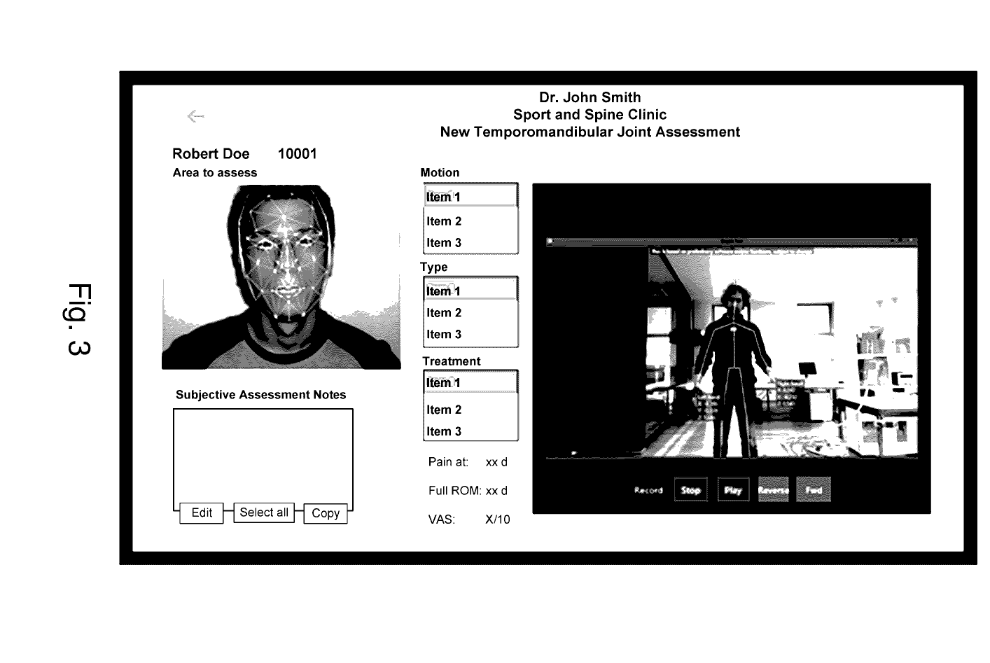

Reference is now made to FIG. 1 which is a flow chart showing a first exemplary computer-implemented method 100 for assessing a human subject's range of motion movements relating to their jaws and jaw muscles. The method 100 is carried out by a data processing system using information received from a 3D infrared camera, a microphone, and a bite force meter, all in communication with a microprocessor. At step 102, a panel shown by way of example (FIG. 2) is provided on a screen communicating with the microprocessor for a practitioner to input their file information, the subject's information, and to position the 3D infrared camera to capture the subject's target facial points, i.e., the eyes, nose, mouth, ears, cheeks, forehead, jaw, and neck. Step 104 determines if the subject has properly positioned their target facial points in front of the 3D infrared camera. If the answer is no, then step 106 is a prompt provided by the computer-implemented method to instruct the subject to adjust their position in front of the camera. If the answer is yes, then step 108 creates an avatar with a set of defined target facial points (step 110) and provides an exemplary panel for the practitioner to fix a screenshot of the target headshot and enter the parameters and criteria for the range of motion movement that will be recorded during the subject's performance of the specified movements, which in this example, are repetitive spaced-apart biting forces applied to the bite force meter (FIG. 3). Steps 112, 114, 116, 118, 120 are carried out substantially in order while the subject applies a series of repetitive spaced-apart biting forces applied to the bite force meter for a set period of time while video imagery is recorded concurrently with data from the bite force meter and audio comments from the subject. During the subject's performance of the repetitive spaced-apart biting forces, the practitioner is monitoring the data being recorded and makes entry into the avatar panel (FIG. 3) regarding their subjective assessment notes (step 122). At step 124, the practitioner can make a decision to end or alternatively, to continue having the subject make further repetitive spaced-apart biting forces applied to the bite force meter. At the conclusion of the testing period, steps 126 and 128 are carried out to reproduce and store the captured imagery, audio, and biting force records with additional subjective notes from the practitioner for future reference (FIG. 2). At the beginning of the next session to assess the subject's repetitive biting forces, the stored data from the first testing event are used as the reference for positioning the subject in steps 102, 104, 106. It should be noted that the avatar created at steps 108, 110 during the practitioner's first data session with the subject to record their imagery, audio, and physiological data, can used in subsequent sessions with the subject to show cues and prompts to guide the subject with assistance to reproduce the selected range of motion movement in an identical manner to the same movement performed during the first session. It should also be noted that the avatar created from the practitioner's first session with the subject, can be used for facial recognition of the subject for use to automatically open the subject's file for the practitioner.

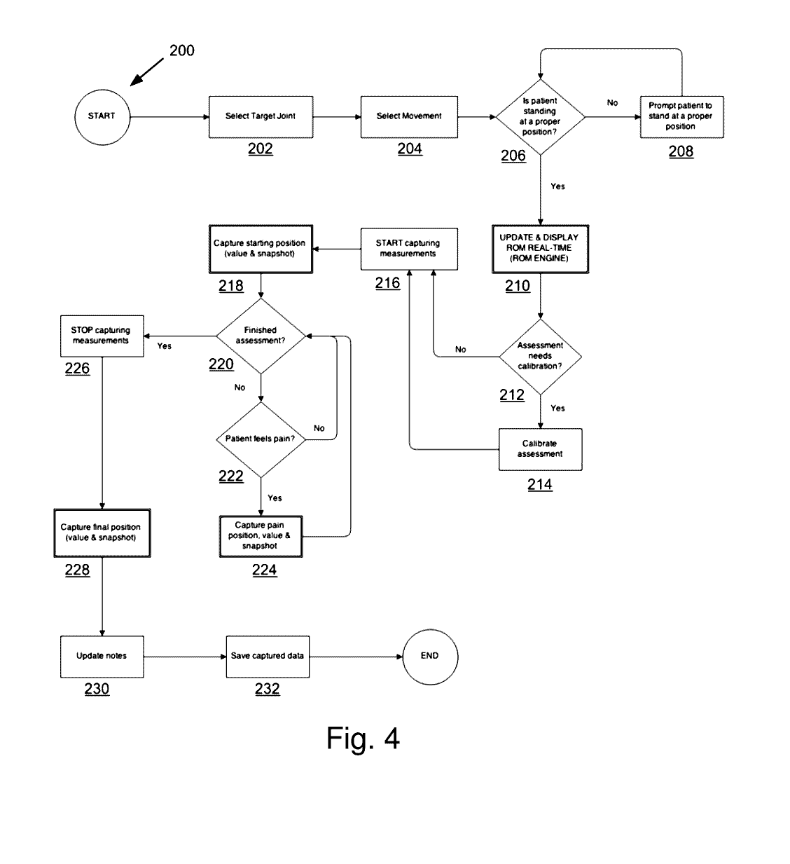

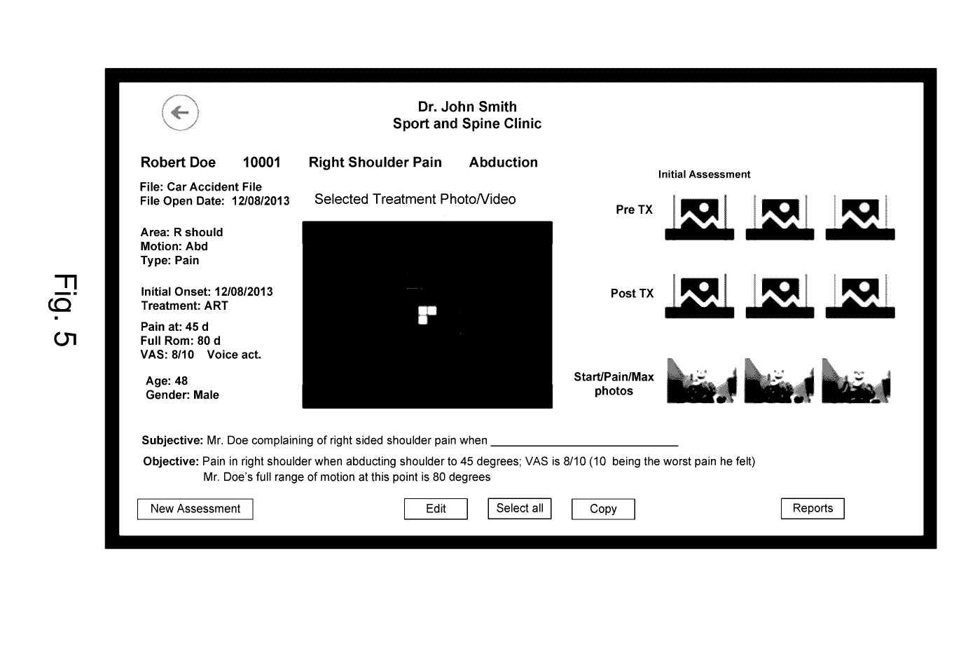

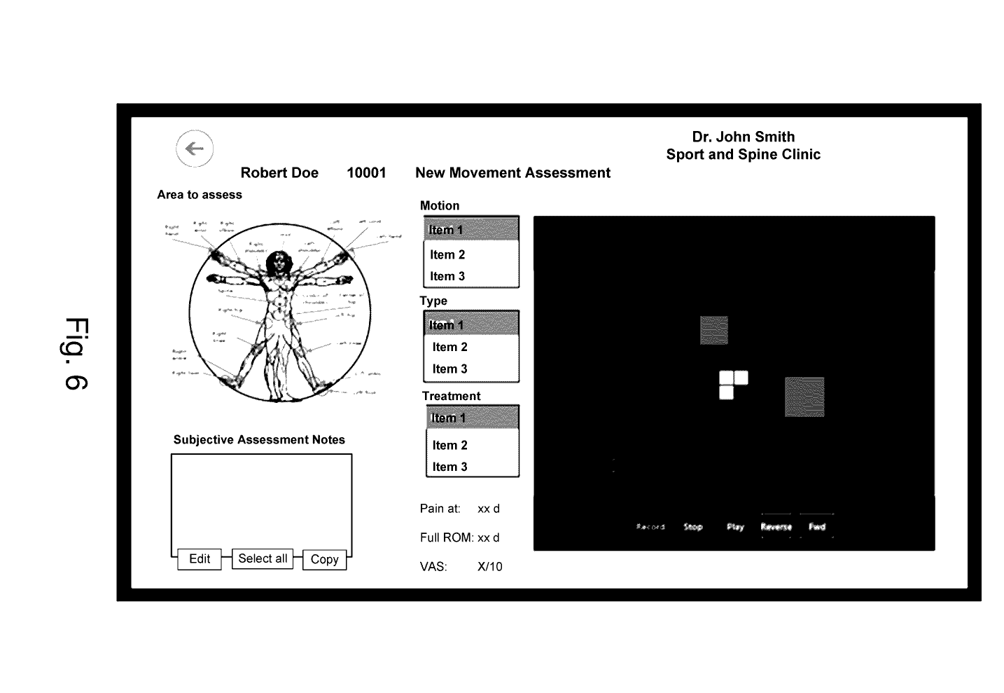

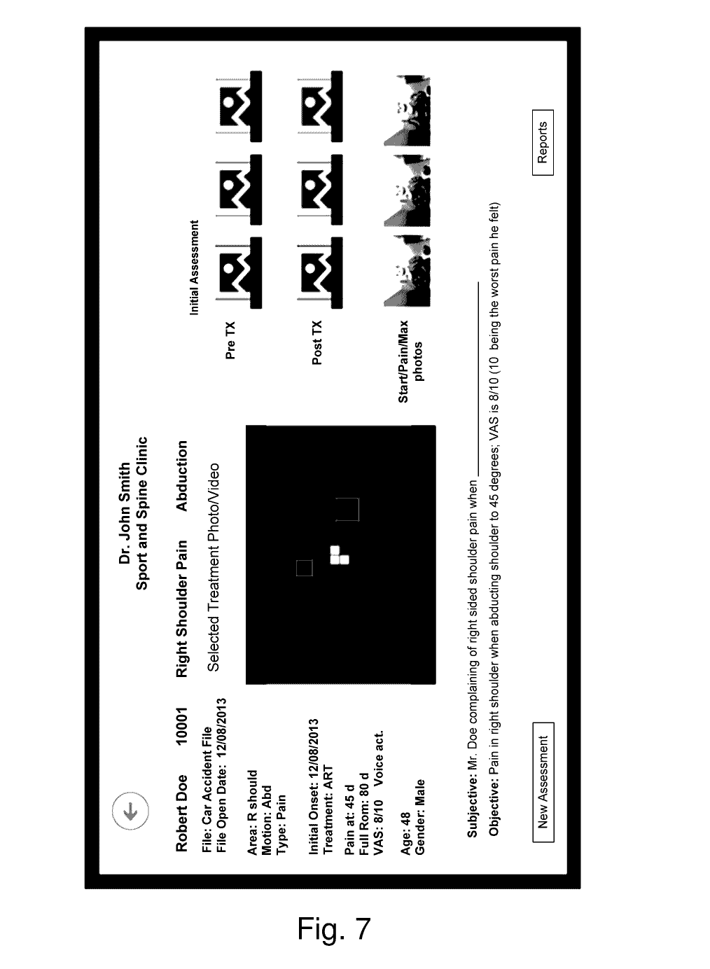

Reference is now made to FIG. 4 which is a flow chart showing another exemplary computer-implemented method 200 for assessing a human subject's range of motion movements relating to an injured body portion resulting from an accident event. The method 100 is carried out by a data processing system using information received from a 3D infrared camera, a microphone, an EMG monitor, a GSR monitor, a heart rate monitor, and a blood pressure monitor, all in communication with a microprocessor. Steps 202, 204, 206, 208, 210, 212, 214 are carried out sequentially concurrent with the practitioner's input into panel such as those exemplified in FIGS. 5-7. In this example, the subject is experiencing significant pain in their right shoulder during performance of routine daily activities, and the practitioner's inputs into the panels exemplified in FIGS. 5-7 define the location and occurrence of pain, and the selected ranges of motion movements that will be performed by the subject. For each selected range of motion movement, steps 218, 220, 222 are then carried out while data is captured from the 3D infrared camera, EMG monitor, a GSR monitor, heart rate monitor, and blood pressure monitor until in the opinion of the practitioner, sufficient data is captured. Steps 226 and 228 are then carried out, after which the practitioner updates their notes into the exemplary panels (step 230) and terminates the session by saving the captured data (step 232). At the beginning of the next session to assess changes in the subject's selected range of motion movements pertaining to their injured body potion, the stored data from the first testing event can be used as a reference for precisely positioning the subject during steps 202, 204, 206, 208, 210, 212, 214, prior to their performance of the selected range of motion movement.

Additionally, it is within the scope of the present disclosure for the practitioner to guide the subject through the performance of a selected practice movement or a selected therapeutic movement following steps 202, 204, 206, 208, 210, 212, 214, and capturing the subject's imagery data during their performance of the selected practice movement or therapeutic movement as a reference selected movement. The computer-implemented method can then provide a series of cues and prompts to guide the subject to reproduce selected practice movement or therapeutic movement in a manner that is identical to their performance of the movement during the session wherein the imagery data for the reference selected movement was captured. It is optional for the practitioner to create a reference avatar with the imagery data captured during the first reproduction of the reference selected movement and provide a set of baseline reference marker locations on the avatar. Imagery data collected from the subject during subsequent sessions can be compared to the location of the baseline reference marker locations in the reference avatar, and used to illustrate for the subject, their progress made in performance of the selected range of motion movement.

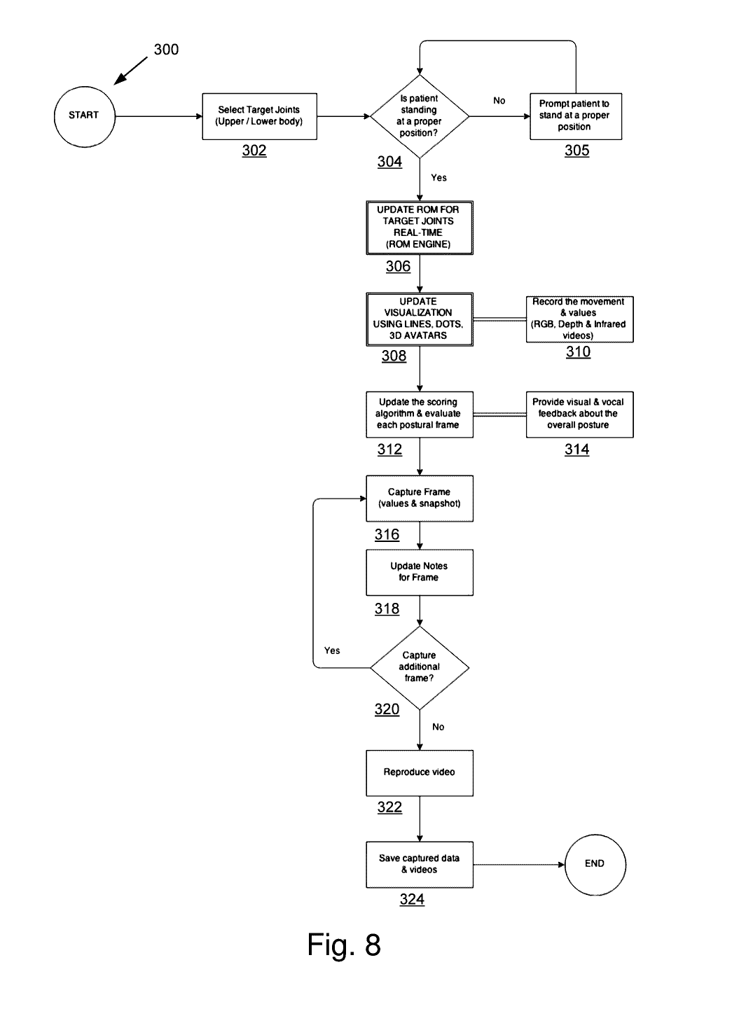

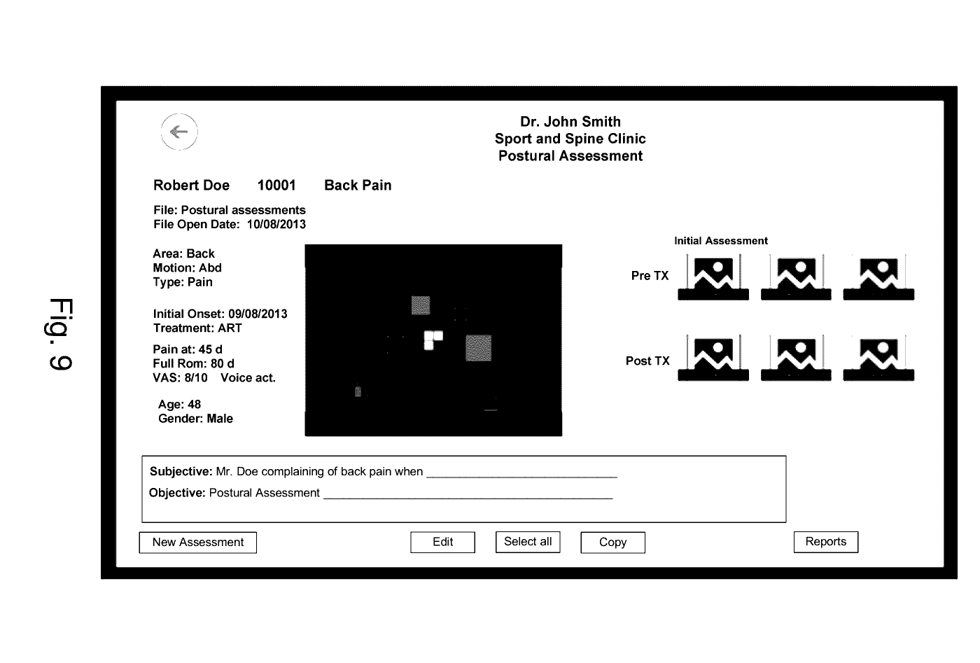

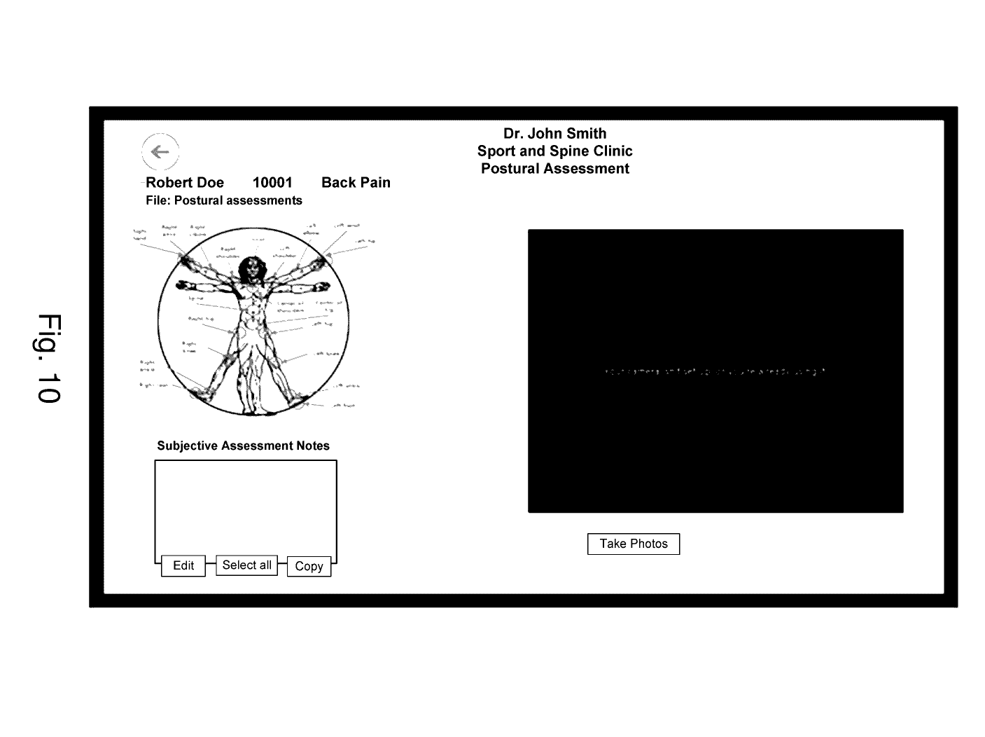

Reference is now made to FIG. 8 which is a flow chart showing another exemplary computer-implemented method 300 for assessing a selected set of range of motion movements related to assisting a human subject to improve their posture. The method 300 is carried out by a data processing system using information received from a 3D infrared camera, a microphone, an EMG monitor, and a GSR monitor, all in communication with a microprocessor. Steps 302, 304, 305, 306 are carried out sequentially concurrent with the practitioner's input into panel such as those exemplified in FIGS. 9-10. After one or more 3D avatars are created (to correspond with the different selected range of motion movements), steps 308, 310, 312, 314, 316, 318, 320 are carried while the practitioner provides inputs into the panels provided by the computer-implemented method 300. After the practitioner is satisfied with the data sets collected, steps 322 and 324 are carried out. At the beginning of the next session to assess changes in the subject's selected range of motion movements pertaining to their posture, the stored data from the first testing event are used as the reference for positioning the subject in steps 302, 304, 305, 306. It is optional for the computer-implemented method to be modified for: (i) creating a reference avatar from the subject's performance of one or more selected reference movements, (ii) capturing video imagery of the subject's posture during their performance of workplace physical movements that are correlated with their reference avatar, and (iii) in occurrences where the subject's to workplace physical movements indicate that their posture becoming physically predisposed to a musculoskeletal stress or strain, providing computer-generated screen prompts to the user with recommendations for performance of one or more body portion specific exercises and/or stretches in order to alleviate or avoid the occurrence of the musculoskeletal stress or strain.

It is within the scope of the present disclosure to modify the computer-implemented methods and related systems for the purpose of monitoring and assessing changes in a human subject's selected range of motion movements during physical training for improving the subject's performance in a selected sport or alternatively, in the execution of a physical task. For example, one or more golf swings, one or more swings with a baseball bat, one or more swings with a racquet, striding movements and/or maneuvers for ice skating, ballet dancing, gymnastic movements, ideal movements for weight lifting, and the like.

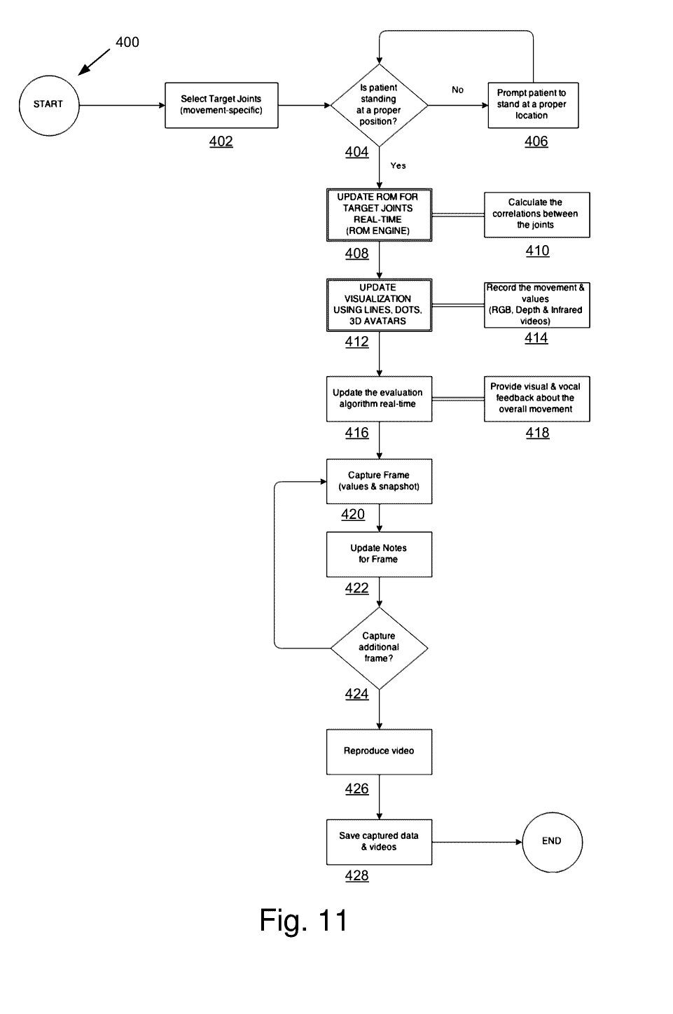

Reference is now made to FIG. 11 which is a flow chart showing another exemplary computer-implemented method 400 for assessing a selected set of range of motion movements related to assisting a subject to improve their golf swing. The method 400 is carried out by a data processing system using information received from a 3D infrared camera, a microphone, and an EMG monitor, all in, with a microprocessor. Steps 402, 404, 406, 408, 410 are carried out sequentially with inputs from a trainer, after which a 3D avatar is generated that defines the location and position of the subject's selected body portions. The subject then performs a series of golf swings during which time steps 412, 414, 416, 418, 420 are sequentially carried out while the trainer inputs their subjective notes (step 422). Step 424 provides the trainer with a decision point to continue with instructions to the subject to make additional golf swings while steps 412, 414, 416, 418, 420, 422 are repeated. Alternatively, the trainer can make the decision to terminate the testing session, after which steps 426 and 428 are carried out. The trainer may use the data and videos captured in step 428 as a teaching tool for instructing the subject on adjustments they need to make to their golf swing mechanics and on a repetitive exercise program to assist their making adjustments to their golf swing mechanics. During the next golf swing training session, the stored data from the first testing event are used as the reference for comparing changes in the subject's golf swing mechanics in the current session with the previous session.

It is also within the scope of the present disclosure to modify the computer-implemented methods and related systems for the purpose of monitoring and assessing changes in animal subject's selected range of motion movements during physical rehabilitation of an injury or an atrophy condition. For example, the computer-implemented methods and related systems are suitable for assessment of gait and posture of animals such as horses and dogs among others. In such applications, the audio data records can be provided by the animal's trainer.

1. A computer-implemented method for assessing changes in a human subject's range of motion for a selected musculoskeletal movement, the method comprising:
(i) positioning the subject to perform a selected musculoskeletal movement;
(ii) arranging at least one 3D infrared video camera to capture a view of a selected portion of the subject's body during their performance of the selected musculoskeletal movement;
(iii) providing a first instruction to the subject to perform the selected musculoskeletal movement and to concurrently provide a verbal commentary when a pain resulting from the movement occurs and/or when a first limit of the movement occurs;
(iv) recording a first video imagery recording of the subject's body portion with the at least one 3D infrared camera during the subject's performance of the selected musculoskeletal movement;
(v) concurrently recording a first audio recording of the subject's verbal commentary;
(vi) concurrently detecting and recording a first data set for a selected physiological parameter during the subject's performance of the selected musculoskeletal movement thereby producing a physiological data recording;
(vii) storing the first visual imagery recording, the audio recording, and the physiological data recording as a first discrete set of data records;
(viii) performing a first kinesiology assessment of the first discrete set of data records;
(ix) preparing a first report summarizing the first kinesiology assessment;
(x) assigning a first unique identification code for the first discrete set of data records, the first kinesiology assessment, and the first report summarizing the first kinesiology assessment;
(xi) after a selected time period, repeating steps (i) to (x) thereby producing a second kinesiology assessment, a second unique identification code and a second report;
(xii) comparing the second kinesiology assessment with the first kinesiology assessment to detect differences during the subject's performance of the selected musculoskeletal movement in one or more of the occurrence of pain, the limit of movement, and ₜhe physiological data; and
(xiii) preparing a progress report summarizing the differences between the first kinesiology assessment and the second kinesiology assessment.

2. The method of claim 1, wherein steps (xi) to (xii) are repeated at one or more additional selected time intervals thereby producing one or more additional kinesiology assessments, and comparing the one or more kinesiology assessments with the first kinesiology assessment to detect differences during the subject's performance of the selected musculoskeletal movement in one or more of the occurrence of pain, the limit of movement, and the physiological data, and preparing one or more additional progress reports summarizing the differences between the first kinesiology assessment and the a one or more additional kinesiology assessments.

3. The method of claim 1, additionally comprising a step of using the progress report to prepare one of a diagnosis, a treatment plan, and an exercise plan.

4. The method of claim 1, wherein the selected physiological parameter is a muscular activity, a heart rate, a blood pressure, an oxygen saturation in the subject's vascular system, a carbon dioxide level in the vascular system, a lactic acid level in the vascular system, a respiration rate, a skin galvanic response, a cardiac activity, a series of eye movements, a change in a dilation of the subject's eyes, or a bite force.

5. A system for cooperating with the computer-implemented method of claim I for assessing changes a human subject's range of motion in response to a physiotherapeutic treatment regime, the system comprising:
at least one 3D infrared camera;
a microphone;
at least one device for receiving and communicating a physiological data set; and
a computer-implemented software program for receiving and processing data inputs from steps (iv), (v), (vi), (xi), and (xii) and generating outputs specified in steps (vii), (viii), (ix), (x), and (xiii).

6. The system of claim 5, wherein the device is one of a heart rate monitor, a blood pressure monitor, a VO2 monitor, a pulse oximeter, a lactic acid monitor, a galvanic skin response monitor, an electrocardiography monitor, a ballistocardiography monitor, an electroencephalography monitor, an electromyography monitor, an electrooculography monitor, and a bite force meter.
